(19) United States
(12) Patent Application Publication
(10) Pub. No.: US 2007/0099883 A1
(43) Pub. Date: May 3, 2007

(54) ANHYDROUS MOMETASONE FUROATE FORMULATION

(76) Inventors: Cheryl Lynn Calis, London, OH (US); Thomas Dean Mahon, West Jefferson, OH (US)

Correspondence Address: PEARNE & GORDON LLP, 1801 EAST 9TH STREET, SUITE 1200, CLEVELAND, OH, 44114-3108, US

(21) Appl. No.: 11/539,769

(22) Filed: Oct. 9, 2006

Related U.S. Application Data
(60) Provisional application No. 60/725,059, filed on Oct. 7, 2005.
(60) Provisional application No. 60/727,334, filed on Oct. 17, 2005.

(51) Int. Cl. A61K 31/58

(52) U.S. Cl. 514/172

(57) ABSTRACT

A stable pharmaceutical composition of anhydrous mometasone furoate, and methods for its preparation, are described.

### CONTINUING APPLICATION DATA

The present application claims priority from U.S. Provisional Application No. 60/725,059 filed Oct. 7, 2005 and U.S. Provisional Application No. 60/727,334, filed Oct. 17, 2005, the disclosures of which are hereby incorporated by reference herein.

### FIELD OF THE INVENTION

The invention is directed to a stable aqueous formulation of anhydrous mometasone furoate.

### BACKGROUND OF THE INVENTION

Mometasone furoate is a corticosteroid that is useful for the treatment of inflammatory conditions. It is primarily administered to treat itching and other inflammatory skin conditions such as eczema, dermatitis, rashes, insect bites, poison ivy, allergies and other irritations, or for upper and lower airway inflammatory conditions such as asthma and allergic or non-allergic rhinitis. While the precise mechanism of mometasone furoate is not currently known, corticosteroids are known to have a wide range of effects on various cell types (e.g., mast cells, eosinophils, neutrophils, macrophages and lymphocytes) and mediators (e.g., histamine, eicosanoids, leukotrienes, and cytokines) involved in inflammation.

Mometasone furoate (9α,21-dichloro-16α-methyl-1,4-pregnadiene-11β,17α-diol-3,20-dione-17-(2′-furoate)) can be prepared according to the procedure disclosed in U.S. Pat. No. 4,472,393. However, U.S. Pat. No. 6,127,353 disclosed that when aqueous pharmaceutical compositions, e.g., suspensions, containing anhydrous mometasone furoate were subjected to stability testing, formation of a crystalline material that was different from anhydrous mometasone furoate crystal was observed. To avoid this undesirable crystal growth during storage, the use of mometasone furoate monohydrate is described as being useful for pharmaceutical compositions, particularly aqueous suspensions where it was thought that its use would reduce the probability of crystal growth during long term storage of the suspension, leading to a more stable product.

### SUMMARY OF THE INVENTION

The present invention provides a stable aqueous anhydrous mometasone furoate suspension and a process of manufacturing this suspension into a pharmaceutically acceptable formulation that uses anhydrous mometasone furoate as the active ingredient and achieves stability in the anhydrous form under long-term storage. One advantage to the use of the anhydrous form of mometasone furoate is that no further processing of the anhydrous mometasone furoate, prepared by standard mometasone furoate synthetic procedures, is needed. Furthermore, it is not necessary to calculate the conversion of monohydrate to the anhydrous form for dosage purposes. The preparation of a stable anhydrous mometasone furoate suspension is unexpected in light of the teachings of U.S. Pat. No. 6,127,353, which indicated that an anhydrous mometasone furoate suspension was unstable when kept under conditions of long-term storage.

Accordingly, the present invention provides a stable pharmaceutical composition comprising anhydrous mometasone furoate in an aqueous pharmaceutically acceptable carrier. In one embodiment, the composition is stable when stored under conditions of about 38° C. to about 42° C. for 3 months. In a further embodiment, the composition is stable when stored under conditions of about 15° C. to about 30° C. for 24 months.

Embodiments of the stable anhydrous mometasone furoate composition include those in which the aqueous pharmaceutically acceptable carrier comprises one or more pharmaceutical excipients selected from the group consisting of suspending agents, humectants, buffer substances, surfactants, and preservatives. In a further embodiment, the stable anhydrous mometasone furoate composition comprises: (a) from about 0.01% to about 1% w/w anhydrous mometasone furoate; (b) from about 0.1% to about 10% w/w suspending agent; (c) from about 0.1% to about 30% w/w humectant; (d) from about 0.001% to about 2% w/w buffer substance; (e) from about 0.001% to about 10% w/w surfactant; (f) from about 0.002% to about 0.5% w/w preservative; and (g) purified water such that the total weight of (a) through (g) is 100%.

In an embodiment of the stable anhydrous mometasone furoate composition including specified proportions of excipients described above, the suspending agent comprises microcrystalline cellulose and sodium carboxymethylcellulose. In a further embodiment, the humectant comprises glycerin. In another embodiment, the buffer substance comprises citric acid and sodium citrate. In yet another embodiment, the surfactant comprises polyoxyethylene 20 sorbitan monooleate. In a further embodiment, the preservative comprises benzalkonium chloride. Yet another embodiment of the stable anhydrous mometasone furoate composition comprises: (a) from about 0.01% to about 1% w/w anhydrous mometasone furoate; (b) from about 0.1% to about 10% w/w combined microcrystalline cellulose and sodium carboxymethylcellulose; (c) from about 0.1% to about 30% w/w glycerin; (d) from about 0.001% to about 2% w/w combined citric acid and sodium citrate; (e) from about 0.001% to about 10% w/w polyoxyethylene 20 sorbitan monooleate; (f) from about 0.002% to about 0.5% w/w benzalkonium chloride; and (g) purified water such that the total weight of (a) through (g) is 100%.

Embodiments of the stable anhydrous mometasone furoate composition include those in which the anhydrous mometasone furoate comprises micronized anhydrous mometasone furoate. Further embodiments including micronized anhydrous mometasone furoate are formulated for delivery to the upper or lower airway, and yet further embodiments are formulated as a nasal spray.

The present invention also provides a method of preparing the stable aqueous anhydrous mometasone furoate composition that comprises: (a) from about 0.01% to about 1% w/w anhydrous mometasone furoate; (b) from about 0.1% to about 10% w/w suspending agent; (c) from about 0.1% to about 30% w/w humectant; (d) from about 0.001% to about 2% w/w buffer substance; (e) from about 0.001% to about 10% w/w surfactant; (f) from about 0.002% to about 0.5% w/w preservative; and (g) purified water such that the total weight of (a) through (g) is 100%. This method includes the steps of: a) combining the suspending agent, the humectant, and the buffer substance with water to form a suspension; b) combining the surfactant and the anhydrous mometasone furoate with water to form a suspension; c) combining the suspension of step a) and the suspension of step b); d) combining the preservative with water and combining with the suspension of step c); and e) providing sufficient purified water to give the composition the desired weight or volume.

In an embodiment of the method described above, the suspending agent comprises microcrystalline cellulose and sodium carboxymethylcellulose, the humectant comprises glycerin, the buffer substance comprises citric acid and sodium citrate, the surfactant comprises polyoxyethylene 20 sorbitan monooleate, and the preservative comprises benzalkonium chloride. In a further embodiment of the method, the composition is stable when stored under conditions of about 38° C. to about 42° C. for 3 months, while in yet another embodiment of the method the composition is stable when stored under conditions of about 15° C. to about 30° C. for 24 months.

The above summary of the present invention is not intended to describe each disclosed embodiment or every implementation of the present invention. The description that follows more particularly exemplifies illustrative embodiments.

### DETAILED DESCRIPTION OF EMBODIMENTS OF THE INVENTION

The invention provides compositions and methods of preparing stable aqueous suspensions of anhydrous mometasone furoate. In one aspect, the invention provides a stable pharmaceutical composition comprising anhydrous mometasone furoate in an aqueous pharmaceutically acceptable carrier. Mometasone furoate (9α,21-dichloro-16α-methyl-1,4-pregnadiene-11β,17α-diol-3,20-dione-17-(2′-furoate)) is a corticosteroid with anti-inflammatory activity that may be prepared according to, for example, the procedure described in U.S. Pat. No. 4,472,393. Mometasone furoate prepared in this fashion is prepared as the non-hydrated corticosteroid and is referred to herein as anhydrous mometasone furoate, or in the alternative, as mometasone furoate anhydrous, and has a molecular formula of C₂₇H₃₀Cl₂O₆. Anhydrous mometasone furoate is mometasone furoate that lacks an associated water molecule. Anhydrous mometasone furoate can be distinguished from mometasone furoate monohydrate, which is a hydrated form of mometasone furoate that has a molecular formula of C₂₇H₃₀Cl₂O₆H₂O.

The stable pharmaceutical composition comprising anhydrous mometasone furoate is provided in an aqueous pharmaceutically acceptable carrier. In accordance with the invention, the aqueous anhydrous mometasone furoate composition of the invention is stable over time. A stable pharmaceutical composition is one that possesses both chemical and physical stability over a specified period of time. A pharmaceutical composition with chemical stability is one in which the active ingredient does not substantially degrade over time, but rather retains its chemical form and activity. For example, a pharmaceutical composition with chemical stability is one in which the pharmaceutical activity of the active ingredient does not vary by more than 10% from its original activity. Similarly, a pharmaceutical composition with physical stability is one in which the physical properties of the composition do not substantially change over the relevant time period. Examples of physical properties of a pharmaceutical composition include, for example, viscosity, pH, density, and suspension homogeneity. Examples of deviation of these physical properties that do not represent a substantial change for the pharmaceutical composition of the invention include viscosity ranging from 50-200 centipoise (cps), with respect to a preferred viscosity of 100 cps, pH ranging from 4.3 to 4.9 with respect to a preferred pH of 4.6, and density ranging from 1.00-1.04 grams/milliliter (g/mL) with respect to a preferred density of 1.014 g/mL. When visually inspected, the pharmaceutical composition is generally an even white to off-white suspension. As a homogenous suspension, the suspension should not exhibit an uneven distribution of materials within the suspension. For example, the formation of layering and/or crystal growth within the suspension both represent deviations from suspension homogeneity. Visual inspection of the pharmaceutical composition of the invention demonstrates no significant layering and/or crystal growth during long term storage of the stable composition.

In accordance with the invention, the pharmaceutical composition remains stable when stored for specified amounts of time and temperature. For example, embodiments of the invention include pharmaceutical compositions that remain stable when stored for about 24 months at a temperature in the range of about 15° C. to about 30° C. In further embodiments, the pharmaceutical composition remains stable when stored for 3 months at a temperature in the range of about 38° C. to about 42° C. In each case, the suspension remains stable during the entire period of storage. Storage for 24 months or more is defined herein as “long-term storage.” In further embodiments, the relative humidity during storage of the stable pharmaceutical composition may also be specified. For example, the pharmaceutical composition may be stable when stored for 3 months at a temperature of about 38° C. to about 42° C. and a relative humidity of 70% to 80%. During storage, the pharmaceutical composition is preferably protected from evaporation and substantial exposure to light. This may be accomplished, for example, by storage of the pharmaceutical composition in a sealed, opaque bottle made of chemically inert material such as high-density polyethylene.

### Formulation of Anhydrous Mometasone Furoate

The invention provides a stable pharmaceutical composition comprising anhydrous mometasone furoate in an aqueous pharmaceutically acceptable carrier. In addition to water, the aqueous pharmaceutically acceptable carrier may include one or more pharmaceutical excipients. Pharmaceutical excipients provided in the pharmaceutical composition may include, inter alia, suspending agents, humectants, buffer substances, surfactants, and preservatives.

The stable pharmaceutical composition of the invention may include varying percentages of anhydrous mometasone furoate and various types of one or more pharmaceutical excipients. For example, in one embodiment of the invention, the composition includes from about 0.01% to about 1% w/w anhydrous mometasone furoate; from about 0.1% to about 10% w/w suspending agent; from about 0.1% to about 30% w/w humectant; from about 0.001% to about 2% w/w buffer substance; from about 0.001% to about 10% w/w surfactant; from about 0.002% to about 0.5% w/w preservative; and the balance being purified water.

The active ingredient, anhydrous mometasone furoate, may be provided in an amount from about 0.01% to about 1% (w/w) of the aqueous composition. For certain embodiments of the invention, the anhydrous mometasone furoate will be provided in a micronized particle form. The particle sizes used for the anhydrous mometasone furoate are those that are conventional in the art for corticosteroids, particularly those used in nasal formulations. Exemplary conventional particle sizes for corticosteroids used in suspensions are as follows: from about 5 to about 10 microns; less than about 5 microns; less than about 4 microns; less than about 2 microns; and less than about 1 micron. In a typical embodiment, the mean particle size of the mometasone furoate is from about 1 micron to about 3 microns.

The stable pharmaceutical composition may also include a suspending agent. Suspending agents help reduce the sedimentation rate of particles in a suspension, and are useful for suspending, for example, insoluble particles that are dispersed in a liquid vehicle. Suspending agents generally function by increasing the viscosity of the liquid vehicle, and thereby slowing settling. The stable pharmaceutical composition of the invention will typically include from about 0.1% to about 10% (w/w) of a suspending agent, with an amount of about 2% (w/w) being preferred. Examples of suspending agents suitable for use in the pharmaceutical composition include microcrystalline cellulose and sodium carboxymethylcellulose, hydroxypropyl-methylcellulose, calcium carboxymethylcellulose, sodium alginate, hydroxyethyl cellulose, hydroxyethylmethyl cellulose, hydroxypropyl cellulose, xanthan gum, guar gum, acacia, tragacanth, carrageenan, and carbomer. Preferably, the pharmaceutical composition includes Avicel®RC-591 as a suspending agent. Avicel®RC-591 is a trademark for a mixture of microcrystalline cellulose and sodium carboxymethylcellulose. Preferably, the Avicel®RC-591 mixture includes about 11% w/w sodium carboxymethylcellulose.

The stable pharmaceutical composition may also include a humectant. Humectants are hygroscopic pharmaceutical excipients that help retain moisture. For example, humectants used in a nasal formulation may help maintain moisture levels in nasal mucosa. The stable pharmaceutical composition of the invention will typically include from about 0.1% to about 30% (w/w) of a humectant, with an amount of about 2% (w/w) being preferred. Examples of humectants suitable for use in the pharmaceutical composition include glycerin, propylene glycol, xylitol, sorbitol, triacetin, mineral oil, polydextrose, and triethanolamine. Preferably, the pharmaceutical composition includes glycerin as the humectant.

The stable pharmaceutical composition may also include one or more buffering substances. A buffering substance (also referred to herein as a buffer) is a weak acid or base that may be added to a solution to provide the solution with the ability to resist changes in pH and/or provide the solution with a particular pH. A buffered solution contains either a weak acid and its conjugate base, or a weak base and its conjugate acid. Preferably, the buffer maintains the pharmaceutical composition of the invention at a pH in the range of about 3 to about 6. More preferably, the buffer maintains the pharmaceutical composition at a pH in the range of about 4.3 to about 4.9. The stable pharmaceutical composition of the invention will typically include from about 0.001% to about 2% (w/w) of a buffer, with an amount of about 0.5% (w/w) being preferred. Examples of buffer substances suitable for use in the pharmaceutical composition include the acids citric acid, malic acid, tartaric acid, acetic acid, and succinic acid and the bases sodium citrate, sodium borate, potassium citrate, sodium phosphate, sodium acetate, and sodium succinate. Preferably, the pharmaceutical composition includes a combination of citric acid monohydrate and sodium citrate dihydrate as buffer substances.

The stable pharmaceutical composition may also include one or more surfactants. Surfactants are typically amphipathic organic compounds that act as wetting agents. The stable pharmaceutical composition of the invention will typically include from about 0.001% to about 10% (w/w) of a surfactant, with an amount of about 0.01% (w/w) being preferred. Examples of surfactants suitable for use in the pharmaceutical composition include sorbitan esters such as sorbitan fatty acid esters, polyoxyethylene sorbitan fatty esters including polysorbates of various molecular weights, polyethylene glycol, sodium lauryl sulfate, polyoxyethylene castor oil derivatives, docusate sodium, poloxamer, polyoxyethylene alkyl ethers, and polyoxyethylene stearates. Preferably, the pharmaceutical composition includes polysorbate 80, which is a trade name for polyoxyethylene 20 sorbitan monooleate, or (Z)-sorbitan mono-9-octadecenoate poly(oxy 1,2-ethanediyl) derivatives.

The stable pharmaceutical composition may also include one or more preservatives. A preservative is a substance that helps to control or inhibit the growth of microorganisms and/or fungi in a pharmaceutical composition. The stable pharmaceutical composition of the invention will typically include from about 0.002% to about 0.5% (w/w) of a preservative, with an amount of about 0.04% (w/w) being preferred. Examples of preservatives suitable for use in the pharmaceutical composition include benzalkonium chloride, benzenthonium chloride, benzoic acid (e.g., sodium benzoate), methylparaben, propylparaben, sorbic acid, potassium sorbate, benzyl alcohol, butylparaben, ethylparaben, penoxyethanol, phenylethyl alcohol, and EDTA. Preferably, the pharmaceutical composition includes benzalkonium chloride.

In a preferred embodiment of the invention, the stable aqueous pharmaceutical composition of anhydrous mometasone furoate includes from about 0.01% to about 1% w/w anhydrous mometasone furoate; from about 0.1% to about 10% w/w combined microcrystalline cellulose and sodium carboxymethylcellulose; from about 0.1% to about 30% w/w glycerin; from about 0.001% to about 2% w/w combined citric acid and sodium citrate; from about 0.001% to about 10% w/w Polysorbate 80; from about 0.002% to about 0.5% w/w benzalkonium chloride; and the balance being purified water.

Other suitable pharmaceutical excipients useful in formulations and dosage forms according to the invention will be apparent to those of ordinary skill in the art in view of the present disclosure. The stable pharmaceutical formulations according to the invention are suitable for delivery to the upper or lower airway; e.g., for use as a nasal spray.

### Method of Preparing the Stable Aqueous Anhydrous Mometasone Furoate Composition

The aqueous suspension compositions of the present invention may be prepared by mixing anhydrous mometasone furoate with purified water and other pharmaceutically acceptable excipients. In one embodiment, the method includes the following steps. First, the suspending agent and the humectant are dispersed in water. Next, the buffer substance is dissolved in water and added to the aqueous suspension containing the suspending agent and the humectant. A surfactant is then dissolved in water and anhydrous mometasone furoate is added to form a slurry. This slurry is then added to the buffered suspension previously prepared. A preservative is then dissolved in water and mixed into the anhydrous mometasone furoate suspension. Finally, a sufficient weight of purified water is added to obtain the desired volume of suspension. Note that the excipients, water, and anhydrous mometasone furoate may be combined in different orders without departing from the scope of the invention. However, it is preferable to add surfactant and preservative later if possible as these excipients are prone to foaming upon mixing. It is also preferable to add the surfactant before adding the anhydrous mometasone furoate, as the surfactant acts as a wetting agent to facilitate suspension of the active agent. Once prepared, the aqueous anhydrous mometasone furoate suspension is preferably stored in a sealed, opaque container until used. For example, the aqueous suspension may be stored in opaque plastic bottles formed from high-density polyethylene.

### Treatment of Upper or Lower Airway Inflammation

Anhydrous mometasone furoate may be administered to a patient to treat a variety of inflammatory conditions of the upper and lower airway. Examples of such conditions include asthma and allergic and non-allergic rhinitis. Anhydrous mometasone furoate may be administered, for example, by oral inhalation or intranasally to treat diseases of the upper and lower airway. Devices useful for providing appropriate dosages of anhydrous mometasone furoate to the upper and lower airway include nebulizers, inhalers, and pump spray bottles. Appropriate devices for administration to the upper or lower airway are known to those skilled in the art.

In one embodiment, anhydrous mometasone furoate may be administered in specific, measured amounts in the form of an aqueous suspension by use of a pump spray bottle. As known to those skilled in the art, the amount of anhydrous mometasone furoate administered and the treatment regimen used will be dependent on the age, sex and medical history of the patient being treated, the severity of the disease condition and the tolerance of patient to the treatment regimen as evidenced by local toxicity (e.g., nasal irritation and/or bleeding) and by systemic side-effects (e.g. cortisol level).

For the treatment of allergic or non-allergic rhinitis and/or inflammatory diseases of the upper or lower airway passages, the amount of anhydrous mometasone furoate that may be administered as an aqueous suspension may be in the range of about 10 to 5000 micrograms (mcg)/day, 10 to 4000 mcg/day, 10 to 2000 mcg/day, 25-1000 mcg/day, 25 to 400 mcg/day, 25-200 mcg/day, 25-100 mcg/day or 25-50 mcg/day in single or divided doses. For any route of administration, divided or single doses may be used. For example, if a plastic nebulizer is used to deliver 200 micrograms a day of an aqueous suspension of anhydrous mometasone furoate, delivery of 50 micrograms twice into each nostril of a patient (providing a total of four doses) could be used to deliver the drug.

In treating allergic and non-allergic rhinitis, the aqueous suspension of anhydrous mometasone furoate may be administered intranasally by inserting an appropriate device (such as a pump spray bottle) into each nostril and then expelling active drug. Further description of the use of mometasone furoate to treat upper and lower airway inflammatory diseases such as asthma and allergic and non-allergic rhinitis can be found in U.S. Pat. No. 6,723,713 or in the 2006 Physicians Desk Reference, Thomson PDR (2005), where the use of mometasone furoate monohydrate is described under the tradename Nasonex®.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Preparation of an Aqueous Anhydrous Mometasone Suspension

A stable aqueous formulation of anhydrous mometasone was prepared. First, Avicel® RC-591 (7.6 kilograms (kg); 2.0% w/w) was dispersed in purified water (230 kg) in a 500 liter stainless steel mixing tank by agitation for 60 minutes at 356 rpms. Glycerin (USP) (7.98 kg; 2.1% w/w) was then added to the dispersed Avicel® RC-591, and the mixture was agitated for 10 minutes at 277 rpms. Citric acid monohydrate (760 grams (g); 0.2% w/w) and sodium citrate dihydrate (1.064 kg; 0.28% w/w) were then dissolved in purified water (7.6 kg) to form a buffer solution in a stockpot provided with an agitator, by agitation at 504 rpms for 10 minutes. The buffer solution was then added to the Avicel® RC-591 and glycerin suspension and agitated for 10 minutes at 277 rpms. Polysorbate 80 (38.0 g; 0.01% w/w) was then dissolved in purified water (12.16 kilograms) in a separate stockpot, again provided with an agitator, and was agitated at 539 rpms for 10 minutes. Anhydrous mometasone furoate (190 g; 0.05% w/w) was then added to the Polysorbate 80 suspension and agitated at 504 rpms for 90 minutes to form a slurry. Care was taken to minimize the exposure to light during addition of the anhydrous mometasone furoate. The slurry containing Polysorbate 80 and the anhydrous mometasone furoate was then added to the previously prepared suspension containing buffer, suspension agent, and humectant, and the combined suspension was agitated at 254 rpms for 10 minutes. Benzalkonium chloride (152 g; 0.04% w/w) was then dissolved in purified water (82 kg) and gently agitated at 71 rpms for 10 minutes in a 180 liter stainless steel tank, and then added to the anhydrous mometasone furoate containing suspension. The combined suspension was agitated at 277 rpms for 10 minutes. Finally, a quantity sufficient (q.s.) of purified water (26 kg) was added to the combined suspension to obtain the desired weight (380 kg), while continuing to agitate the suspension. The suspension was then delivered from the processing tank to a 500 liter stainless steel storage tank through an APV brand homogenizer. An aqueous anhydrous mometasone suspension was thus prepared that contained anhydrous mometasone furoate, excipients, and water in the following amounts:

| Ingredient | Amount (% w/w) |
|---|---|
| Anhydrous mometasone furoate | 0.05% |
| Avicel ® RC-591 | 2.0% |
| Glycerin | 2.1% |
| Citric acid (monohydrate) | 0.2% |
| Sodium citrate (dihydrate) | 0.28% |
| Polysorbate 80 | 0.01% |
| Benzalkonium chloride | 0.04% |
| Purified water | 95.34% |

The lot of anhydrous mometasone furoate was divided into smaller portions for pharmaceutical use.

### Example 2

### Physical Stability of the Anhydrous Mometasone Furoate Suspension

Samples were tested that had the preferred target pH of 4.6, and higher and lower pH samples having pHs of 4.9 and 4.3 respectively. Samples of the lots were analyzed for visual description, viscosity, pH, and density. Samples from the lots were stored in HDPE bottles at 40° C. (+/−2° C.) and 75% relative humidity (RH) (+/−5%) for three months and analyzed. The results are provided below:

| | | Vis- | | |
|---|---|---|---|---|
| | | cosity | | Density |
| Lot | Visual Description | (CPS) | pH | (g/mL) |
| Target pH | White to off-white suspension | 85 | 4.61 | 1.014 |
| Initial | | | | |
| Target pH | White to off-white suspension | 124 | 4.63 | 1.014 |
| 3 month | | | | |
| 40° C./ | | | | |
| 75% RH | | | | |
| Low pH (4.3) | White to off-white suspension | 87 | 4.25 | 1.010 |
| Initial | | | | |
| Low pH (4.3) | White to off-white suspension | 185 | 4.34 | 1.015 |
| 3 month | | | | |
| 40° C./ | | | | |
| 75% RH | | | | |
| High pH (4.9) | White to off-white suspension | 113 | 4.82 | 1.005 |
| Initial | | | | |
| High pH (4.9) | White to off-white suspension | 185 | 4.88 | 1.015 |
| 3 month | | | | |
| 40° C./ | | | | |
| 75% RH | | | | |

These results demonstrate that the anhydrous mometasone furoate suspension is physically stable.

### Example 3

### Optical Microscopy of Anhydrous Mometasone Furoate

Samples of the anhydrous mometasone furoate composition were also examined by optical microscopy in order to determine if the anhydrous mometasone furoate particles within the suspension change size with time. Both the initial sample and the sample after 3 months at 40° C. and 75% relative humidity time points were examined. The samples were placed on a microscope slide using an eye dropper and examined by polarized light microscopy. The examination was conducted between crossed-polars with a first-order red compensator inserted into the optical path. Anhydrous mometasone furoate particles were identified according to their optical crystallographic properties. No change in anhydrous mometasone furoate particle size was observed between the initial and 3 month time points.

### Example 4

### X-ray Analysis of Anhydrous Mometasone Furoate Suspension

The stability of the anhydrous mometasone furoate suspension prepared as described in Example 1 was tested using an X-ray powder diffractometer. A Bruker AXS X-Ray Powder Diffractometer Model D8 Advance (Bruker-AXS; Karlsruhe Germany) using CuKa radiation (1.54 Å) in parallel beam mode with a Gobel Mirror, LiF monochromator and a scintillation detector was used to determine if the anhydrous mometasone furoate of the pharmaceutical suspension changed form during storage. Solids were concentrated using a centrifuge run for 5 minutes at 3000 rpm. The wet cake at the bottom of the centrifuge tube was placed on a zero background quartz holder. A detector step scan using a tube angle of 1°2θ was performed. Samples were scanned from 2-35°2θ, using a step size of 0.05°2θ, 4 secs per step at 40 kV, 40 mA. The spacing of the observed diffraction peaks was then calculated.

To obtain stability data, samples of the anhydrous mometasone furoate pharmaceutical composition were stored in sealed, opaque plastic bottles for the time, temperature, and humidity indicated. Samples were tested that had the preferred initial pH of 4.6, and higher and lower pH samples having pHs of 4.9 and 4.3, respectively. Samples were tested by obtaining a portion of the sample and concentrating it to a wet solid as described above, and then testing the sample by X-ray powder diffractometry. The samples were obtained from stored samples, as well as from samples that had not been stored, and the data from these samples was compared to determine if any change of the active ingredient (anhydrous mometasone furoate) had occurred. For all three samples (e.g., pH 4.3, 4.6, and 4.9), the X-ray pattern was substantially similar after storage to the initial sample. In addition, x-ray powder diffractometry was carried out on a sample of Nasonex® mometasone furoate monohydrate. Comparison of this spectra with the spectra obtained from the analysis of the anhydrous mometasone furoate confirmed that different spectra were produced.

### Example 5

### HPLC Analysis of Anhydrous Mometasone Furoate Suspension

The stability of the anhydrous mometasone furoate suspension prepared as described in Example 1 was also tested using high performance liquid chromatography (HPLC). HPLC analysis was conducted using a C18, 3μ, 150 mm×4.6 mm column (Phenomenex Gemini) column and a guard column. A flow rate of 1.1 mL/minute and a run time of 75 minutes were used, with a column temperature of 40° C. and UV detection at 248 nm. The mobile phase consisted of a 59/41 mixture of methanol/0.1% H₃PO₄. Samples were prepared by mixing a portion of the pharmaceutical composition with acetonitrile:H₂O:acetic acid (60:40:0.2), and centrifuging and filtering the sample before delivering a 100 μL sample to the column.

Samples were tested that had the preferred target pH of 4.6, and higher and lower pH samples having pHs of 4.9 and 4.3 respectively. Samples of the lots were analyzed via HPLC initially (0 month) for assay of anhydrous mometasone furoate and degradation products. Samples from the lots were stored in high-density polyethylene (HDPE) bottles at 40° C. (+/−2° C.) and 75% relative humidity (RH) (+/−5%) for three months and analyzed via HPLC for assay of anhydrous mometasone furoate and degradation products. The results are provided below:

| | | Specified | Specified Known | Single Largest | |
|---|---|---|---|---|---|
| | | Unknown | Degradant | Unknown (SLU) | |
| | Assay | Degradant (% w/w) | (% w/w) | Degradant | Total Degradants |
| Lot | (% LA) | @ RRT 0.52 | @ RRT 0.88 | (% w/w) | (% w/w) |
| Target pH | 101.2% | BQL | BQL | 0.0535 | 0.0535 |
| Initial | | | | | |
| Target pH | 102.9% | BQL | BQL | 0.0557 | 0.0557 |
| 3 month | | | | | |
| 40° C./75% RH | | | | | |
| Low pH (4.3) | 99.6% | BQL | BQL | 0.0528 | 0.0528 |
| Initial | | | | | |
| Low pH (4.3) | 100.3% | BQL | 0.0783 | 0.0556 | 0.1339 |
| 3 month | | | | | |
| 40° C./75% RH | | | | | |
| High pH (4.9) | 100.1% | BQL | BQL | 0.0534 | 0.0534 |
| Initial | | | | | |
| High pH (4.9) | 100.2% | BQL | 0.0984 | 0.0562 | 0.1546 |
| 3 month | | | | | |
| 40° C./75% RH | | | | | |

RRT = relative retention time
% LA = % labeled amount
BQL = below quantitative limit

The assay provides the activity or potency of the active pharmaceutical ingredient reported as a percent of the labeled or theoretical amount before and after storage at 3 months 40° C./75% RH. The assay for degradants provides a measure of the amount of degradation of the pharmaceutical ingredient, reported as a percentage of the degradant compared to the active ingredient. The HPLC data indicate that the pharmaceutical composition containing the anhydrous form of mometasone fuorate is chemically stable with respect to assay and degradation products and that the manufacturing process utilized produces a substantially stable product.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

What is claimed is:

1. A stable pharmaceutical composition comprising anhydrous mometasone furoate in an aqueous pharmaceutically acceptable carrier.

2. The stable pharmaceutical composition of claim 1, wherein the composition is stable when stored under conditions of about 38° C. to about 42° C. for 3 months.

3. The stable pharmaceutical composition of claim 1, wherein the composition is stable when stored under conditions of about 15° C. to about 30° C. for 24 months.

4. The stable pharmaceutical composition of claim 1, wherein the aqueous pharmaceutically acceptable carrier comprises one or more pharmaceutical excipients selected from the group consisting of suspending agents, humectants, buffer substances, surfactants, and preservatives.

5. The stable pharmaceutical composition of claim 4, wherein the composition comprises:
(a) from about 0.01% to about 1% w/w anhydrous mometasone furoate;
(b) from about 0.1% to about 10% w/w suspending agent;
(c) from about 0.1% to about 30% w/w humectant;
(d) from about 0.001% to about 2% w/w buffer substance;
(e) from about 0.001% to about 10% w/w surfactant;
(f) from about 0.002% to about 0.5% w/w preservative; and
(g) purified water such that the total weight of (a) through (g) is 100%.

6. The stable pharmaceutical composition of claim 5, wherein the suspending agent comprises microcrystalline cellulose and sodium carboxymethylcellulose.

7. The stable pharmaceutical composition of claim 5, wherein the humectant comprises glycerin.

8. The stable pharmaceutical composition of claim 5, wherein the buffer substance comprises citric acid and sodium citrate.

9. The stable pharmaceutical composition of claim 5, wherein the surfactant comprises polyoxyethylene 20 sorbitan monooleate.

10. The stable pharmaceutical composition of claim 5, wherein the preservative comprises benzalkonium chloride.

11. The stable pharmaceutical composition of claim 4, wherein the composition comprises:
(a) from about 0.01% to about 1% w/w anhydrous mometasone furoate;
(b) from about 0.1% to about 10% w/w combined microcrystalline cellulose and sodium carboxymethylcellulose;
(c) from about 0.1% to about 30% w/w glycerin;
(d) from about 0.001% to about 2% w/w combined citric acid and sodium citrate;
(e) from about 0.001% to about 10% w/w polyoxyethylene 20 sorbitan monooleate;
(f) from about 0.002% to about 0.5% w/w benzalkonium chloride; and
(g) purified water such that the total weight of (a) through (g) is 100%.

12. The stable pharmaceutical composition of claim 1, wherein the anhydrous mometasone furoate comprises micronized anhydrous mometasone furoate.

13. The stable pharmaceutical composition of claim 12, wherein the composition is formulated for delivery to the upper or lower airway.

14. The stable pharmaceutical composition of claim 13, wherein the composition is formulated as a nasal spray.

15. A method of preparing the stable aqueous anhydrous mometasone furoate composition of claim 5, comprising:
a) combining the suspending agent, the humectant, and the buffer substance with water to form a suspension;
b) combining the surfactant and the anhydrous mometasone furoate with water to form a suspension;
c) combining the suspension of step a) and the suspension of step b);
d) combining the preservative with water and combining with the suspension of step c); and
e) providing sufficient purified water to give the composition the desired weight or volume.

16. The method of claim 15, wherein the suspending agent comprises microcrystalline cellulose and sodium carboxymethylcellulose, the humectant comprises glycerin, the buffer substance comprises citric acid and sodium citrate, the surfactant comprises polyoxyethylene 20 sorbitan monooleate, and the preservative comprises benzalkonium chloride.

17. The method of claim 15, wherein the composition is stable when stored under conditions of about 38° C. to about 42° C. for 3 months.

18. The method of claim 15, wherein the composition is stable when stored under conditions of about 15° C. to about 30° C. for 24 months.
